(19) United States
(12) Patent Application Publication
(10) Pub. No.: US 2005/0096655 A1
(43) Pub. Date: May 5, 2005

(54) Apparatus for the osteosynthesis of bone fractures by means of locked endomedullary nailing

(76) Inventor: Luciano Trinchese, Riccione (IT)

Correspondence Address: PEARNE & GORDON LLP, 1801 EAST 9TH STREET, SUITE 1200, CLEVELAND, OH, 44114-3108, US

(21) Appl. No.: 10/698,918

(22) Filed: Oct. 31, 2003

(51) Int. Cl. A61B 17/58

(52) U.S. Cl. 606/62

(57) ABSTRACT

In an apparatus for the osteosynthesis of bone fractures by means of locked endomedullary nailing, a tubular nail (1), able to be inserted in a medullar channel (3) of a bone (4), has a coaxial through internal channel (2) and has transverse through holes (5a, 5b, 5c, 5d) whose axis (6a, 6b, 6c, 6d) intersects the axis (7) of the nail (1). A device for locating the axis (6a) of a predetermined hole (5a) selected between said holes (5a, 5b, 5c, 5d), along which the bone (4) is to be drilled to drive a corresponding screw for locking the nail (1) on the bone (4), comprises a source (8) of electromagnetic power, an emitter (9) of the electromagnetic power in the form of non ionising electromagnetic radiation, a line (15) for transmitting the electromagnetic power from the source (8) to the emitter (9). The emitter (9) can be inserted inside the nail (1) along the internal channel (2), from a proximal end (10) of the nail (1) at least to a working position, located on the axis (6a) of the predetermined hole (5a), in which at least part of the non ionising electromagnetic radiation is directed from the emitter (9), through the predetermined hole (5a), on an inner superficial portion (11) of the cortex (12) of the bone (4) corresponding to the bone (6a) of the predetermined hole (5a) and generates, beyond an outer superficial portion (13) of the cortex (12) of the bone (4), also corresponding to the axis (6a) of the predetermined hole (5a), a signal (14) detectable from the exterior having intensity distribution with centroid in correspondence with the axis (6a) of the predetermined hole (5a).

### BACKGROUND OF THE INVENTION

The present invention relates to an apparatus for the osteosynthesis of bone fractures by means of locked endomedullary nailing.

Locked endomedullary nailing is used in orthopaedic-traumatological surgery, in particular in the treatment of unstable (or complex or comminuted) diaphyseal fractures of long bones, requiring locked osteosynthesis.

All known apparatuses of this kind comprise an endomedullary nail, usually made of steel. It is generally derived from a tubular model with substantially cylindrical symmetry, coaxially defining a through internal channel for the insertion of the nail itself on a so-called “guide wire”.

In proximity to its tip, also known as “distal end”, the endomedullary nail has two through holes, also known as “distal holes”, obtained transversely to the axis of the nail for the insertion of corresponding distal locking screws (or pins). In proximity to its head, also known as the “proximal end”, the nail also has two similar holes, also known as “proximal holes”, for the insertion of corresponding proximal locking screws (or pins).

The proximal and/or distal holes have a diameter of a few millimetres. The axes of the proximal and distal holes intersect the axis of the nail. The two distal holes have axes that are mutually parallel and perpendicular to the axis of the nail. The proximal holes can have axes variously oriented relative to each other, depending on the type of nail used and on the fracture type.

Given the tubular nature of the nail, each through hole (be it distal or proximal) is reduced to two coaxial circular openings, obtained on the walls of the nail at opposite sides relative to the axis thereof.

Length, outer diameter and, possibly, diameter of the inner channel of the nail vary depending on the type of fracture to be treated.

Some types of nails (generally, those with proximal holes whose axis are not mutually parallel) can be slightly bent back in correspondence with the proximal end.

When treating a fracture, an end of the bone to be recomposed is surgically exposed and an access opening to the endomedullary channel is obtained therein. A thin steel guide wire (possibly, slightly bent at its tip) is then inserted into the access opening, making the guide wire pass inside the portions of endomedullary channel of all the fragments of the fractured bone. This operation is performed with the aid of an X-ray vision system comprising: an emitter of X-rays that irradiates the area of interest from the outside, a corresponding detector located at the opposite side and provided with brilliance amplifier, a viewing screen.

The endomedullary nail, threaded on the guide wire, is then inserted inside the endomedullary channel of the fractured bone, so as to align on itself all the fractured parts of the bone thanks to the action of the guide wire whereon the nail slides. The nail is inserted into the bone nearly to the proximal end, in such a way that the proximal and distal holes are completely inside the bone. This operation is performed manually, generally with the aid of a transverse grip, also known as “hand piece”, which is preventively associated in removable fashion to the proximal end of the nail by means of a fastening pin coaxial to the nail itself. The fastening pin is also provided with an inner channel for the passage of the guide wire. At the end of the insertion of the endomedullary nail, the wire guide is extracted by removing it from the inner channel of the nail.

At this point, the nail must be locked in position by driving the distal and proximal locking screws (or pins) into the bone through the corresponding distal and proximal holes. To obtain the passage for the locking screws, it is necessary to drill the bone from side to side exactly along the axis, respectively, of the proximal holes and of the distal holes (obviously, after surgically uncovering the bone parts in correspondence with which said holes are located). This operation, for which a drilling organ is used (for instance, a drill fitted with a suitable bit), must be performed very carefully.

The problem of driving the locking screws of the nail is very delicate. It is easy to make mistakes in identifying the exact position of the axis of the (proximal or distal) hole of the nail along which the bone must be drilled and, consequently, it is also easy to make mistakes in aligning the drilling organ with the axis of the hole.

A mistake in the bone drilling phase can compromise the stability of the nail implantation and, therefore, the outcome of the surgical operation.

Therefore, usually, known apparatuses for the osteosynthesis of bone fractures by means of locked endomedullary nailing are provided with devices for locating the axis of the hole (distal or proximal) along which the bone must be drilled, which may be provided with accessories for aligning the drilling organ with said axis.

Manual locating devices are known, based on empirical techniques, which provide for the combined use of an X-ray vision system like the one described above and of a simple, thin reference rod, called “Kirschner wire”.

After accurately positioning emitter and detector of the viewing system astride the bone, in such a way that the image of the hole of the nail thus obtained is perfectly circular, the surgeon tries to place the sharp tip of the Kirschner wire in correspondence with the centre of the image of the hole. Once this position is found, keeping the sharp tip still, the Kirschner wire is oriented in such a way that its image, produced by the viewing system, is substantially reduced to a point located approximately in the centre of the image of the hole. In this way, the Kirschner wire and the axis of the hole are approximately aligned. The Kirschner wire is driven slightly into the cortical of the bone, in such a way as to clearly indicate driving point and direction of drilling. Generally, at this point the Kirschner wire is removed and, exploiting the small incision produced thereby as a reference for positioning the drill bit, the perforation is made after trying to position the axis of the drill bit correctly. In performing this latter operation it is very easy to make alignment mistakes, so an alternative solution is not to remove the Kirschner wire and to use it as a physical reference for a tubular drill bit that can be inserted directly on the wire itself used as a guide.

This solution to the alignment problem has very numerous disadvantages, which can be summarised as follows in order of severity.

First of all, the search for the driving point of the locking screws, completely left to the surgeon's skill and manual ability, is complicated and does not guarantee the final result. All operations (positioning the end of the Kirschner wire in correspondence with the hole and subsequent optimisation of the relative position of both, also to determine the direction of the axis of the hole correctly and to allow subsequent alignment of the drilling organ therewith) are performed exposing the treated part to X-rays, whilst the surgeon is forced to monitor the operations through a screen and not by directly viewing them. The times required to install the locking screws are extremely variable in relation to several factors, such as: the skill of the surgeon and of the radiologist, the morphology of the patient and the proper positioning of the X-ray viewing system relative to the part to be treated. Moreover, both the patient and the medical personnel are exposed to a high dose of dangerous ionising radiation during the entire nail locking phase. The time required by involved medical personnel to learn this technique is very long, as it requires a considerable level of manual skill and experience. The complex and delicate nature of the operations to be performed entails a high risk of error.

A recent, modern technique employs appropriate radiographic spatial references which, once the nail is inserted into the endomedullary channel and before the bone is drilled, are placed in various positions of the bone segment to be treated. Subsequently, a series of radiographic images of the bone segment to be treated is taken from various positions and a virtual reality, based on such images, is reconstructed, allowing the surgeon to drill the holes and to position the locking screws of the nail.

This solution to the problem, however, is not free from drawbacks.

Very costly equipment is required, which makes use of said techniques and devices possible practically only in large hospital facilities, which can count on such a number of patients as to allow, in relatively short times, to amortise the acquisition and installation costs of the equipment.

The execution of the operation is in any case complicated and rather laborious. Once again, the time required by medical personnel to learn the technique is long. Moreover, in this case as well, both the patient and the medical personnel are exposed to dangerous ionising radiation during the nail locking phase, albeit to a reduced extent relative to the empirical techniques described above.

Also known are locating devices that use electronic sensors which, made to slide externally on the surface of the bone, are able to detect the difference in impedance due to the characteristics of the endomedullary nail inserted therein. These devices, rarely used today, are not very reliable and they yield occasionally random results. The system is laborious, requires long learning times and has high costs.

Also known are locating devices that use mechanical references of various kinds (sometimes made with materials that are transparent to X-rays) which, positioned externally to the bone, can be made integral with the proximal end of the nail and serve as supports to guides for the bit of the drilling organ and/or for the locking screws. If the mechanical references are positioned correctly relative to the proximal end of the nail, the positions in which the guides are located are, in principle, aligned with those of the proximal and/or distal holes of the nail. The mechanical references are provided with graduated gauges that allow for their correct positioning depending on the length of the nail.

An example of commonly used mechanical reference is a graduated bar that can be made integral with the proximal end of the nail and whereto various accessories can be associated.

Alignment precision thus depends to a considerable extent on the rigidity of the nail-reference system. This rigidity is never absolute, because of the play linked to the coupling of the various mechanical parts and of the intrinsic elasticity of the nail. The nail can flex by effect of the torsion and flexion forces exerted by the mechanical systems used to place the fractured part under traction and/or by effect of the tensions exerted by the soft parts on the nail-reference system.

This problem is aggravated by the fact that the mechanical reference, developing parallel to the axis of the nail, is not fastened in any way to the distal end of the nail itself and thus has a free end. The nail-reference system is therefore “open” in correspondence with the distal end. Near the proximal end, it is sufficiently rigid to minimise deformation and thus assure a sufficient precision in the alignment of the drilling organ with the proximal holes, but it is never so rigid as to prevent, progressively farther away from the proximal end, the relative deformation of the two elements from becoming such as to render uncertain the correspondence between the guides and the holes on the nail. Thus, the device has high probabilities of failure in the driving of the distal screws.

To overcome this problem, the surgeon tries to close the nail-reference system in correspondence with the distal end of the nail by means of a stabiliser rod. The stabiliser rod is mounted at the free end of the mechanical reference in such a way as to be perpendicular to the plane defined by the mutually parallel axes of the distal holes of the nail. It must be brought manually in contact with the proximal end of the nail in correspondence with the axis of the nail itself and maintained there, also manually, during the drilling and distal screw insertion operations.

This solution solves only partially the problem of stiffening the system and it is very complicated, as it requires the intervention of another person, in addition to the surgeon, to keep the stabiliser rod in contact with the nail. Moreover, the stabiliser rod must be partially inserted inside the bone in a position that, in turn, must be determined with sufficient precision. Therefore (obviously, after surgically uncovering this additional part of bone as well) it is necessary to execute an additional partial perforation of the bone perpendicularly to the plane defined by the axes of the distal hole, after correctly aligning the bit of the drilling organ.

In the second place, the use of locating devices provided with mechanical references does not completely eliminate the need to monitor operations using the X-ray vision system. Hence, although to a lesser extent than in the previous techniques, patient and medical personnel are still exposed to dangerous ionising radiation during the nail locking operation.

Moreover, said devices require a complex and costly surgical instrumentation, with consequent difficulties with its use, maintenance, sterilisation and storage.

In general, currently used systems are therefore burdened, depending on the case, by an excessive complexity (often accompanied by a certain degree of imprecision) and/or by an inevitable, and excessive, exposure of the patient and of medical personnel to dangerous radiation sources.

The aim of the present invention is to overcome the above drawbacks, making available an apparatus for the reduction of bone fractures by means of locked endomedullary nailing, which allows simply and without excessive exposure to dangerous radiation to locate with certainty the holes for the insertion of the locking screws.

Another aim of the present invention is to make available an apparatus for the reduction of bone fractures by means of locked endomedullary nailing, which allows to minimise the time required to execute the nail locking operations, whilst assuring a successful outcome.

### SUMMARY OF THE INVENTION

These aims and others besides, which shall become more readily apparent from the description that follows, are achieved, in accordance with the present invention, by an apparatus for the reduction of bone fractures by means of locked endomedullary nailing as described and claimed in the accompanying main claim and in the accompanying dependent claims.

### BRIEF DESCRIPTION OF THE DRAWINGS

The invention is disclosed in greater detail below with the aid of the drawings, which show an embodiment provided purely by way of non limiting example.

FIG. 1 shows a schematic perspective view of a bone with the apparatus of the invention applied thereto.

FIG. 2 shows, partially sectioned in a plane containing the axis of the bone and the axis of a hole, a variation of the apparatus of FIG. 1, with the emitter in working position.

FIG. 3 shows a cross section of the bone and of the nail with a detail of a variation of the apparatus of the invention.

FIGS. 4a, 4b, 4c show an operating principle of the invention together with an additional variation thereof

FIG. 5 shows, partially sectioned in the plane of FIG. 2, a variation of the invention with the emitter in working position.

FIG. 6 shows, partially sectioned in the plane of FIG. 2, a detail of a variation of emitter in working position.

FIGS. 7e 8 respectively show a partial section in the plane of FIG. 2 and in a plane perpendicular thereto of an additional variation of the invention with the emitter in working position.

FIG. 9 shows an exploded view of the assembly comprising a nail, a hand piece and a corresponding coaxial fastening pin.

FIGS. 10a and 10b show a longitudinal section of the coupling area between the nail, hand piece and fastening pin respectively with the pieces separate and with the pieces mutually joined by the fastening pin.

FIGS. 11a and 11b show a cross section of the variation of FIGS. 7 and 8 with the emitter, respectively, away from the working position and in working position.

### DESCRIPTION OF THE PREFERRED EMBODIMENTS

With reference to the figures, an apparatus for the osteosynthesis of bone fractures by means of locked endomedullary nailing comprises a tubular nail 1, which defines a through coaxial internal channel 2. The nail 1, provided with transverse through holes 5a, 5b, 5c, 5d whose axis 6a, 6b, 6c, 6d intersects the axis 7 of the nail 1, can be inserted in a medullar channel 3 of a bone 4. The apparatus further comprises a device for locating the axis 6a of a predetermined hole 5a selected among said holes 5a, 5b, 5c, 5d, axis 6a along which the bone 4 is to be drilled to drive a corresponding screw (not shown herein) for locking the nail 1 on the bone 4.

The locating device comprises a source 8 of electromagnetic power, an emitter 9 of the electromagnetic power in the form of non ionising electromagnetic radiation and a line 15 for transmitting the electromagnetic power from the source 8 to the emitter 9.

The emitter 9 of the electromagnetic power in the form of non ionising electromagnetic radiation can be inserted inside the nail 1 along the internal channel 2. Its travel along the internal channel 2 extends from a proximal end 10 of the nail 1 at least to its work position, located on the axis 6a of the predetermined hole 5a, in which at least part of the non ionising electromagnetic radiation is directed from the emitter 9, through the predetermined hole 5a, on an inner superficial portion 11 of the cortex 12 of the bone 4 corresponding to the axis 6a of the predetermined hole 5a and generates, beyond an outer superficial portion 13 of the cortex 12 of the bone 4, also corresponding to the axis 6a of the predetermined hole 5a, a signal 14 detectable from the exterior and having an intensity distribution with its centroid in correspondence with the axis 6a of the predetermined hole 5a. Obviously, also the transmission line 15 can be inserted at least partially along the internal channel 2, to follow the travel of the emitter 9.

Since the nail 1 is substantially opaque to the radiation exiting the emitter 9, whilst bone tissues are traversed by it (though they may be involved in diffusion and absorption phenomena by the electromagnetic radiation that propagates therein), the signal 14 is generated beyond the outer superficial portion 13 only when the emitter 9 itself is in front of the predetermined hole 5a (or of one of the other holes, be they distal or proximal, into which the locking screws are to be inserted). Since the signal 14 can be detected from the exterior, the hole itself makes itself “visible” to the surgeon. Since the radiation is not ionising, no person present in the operating room is subjected to harmful radiation loads and the surgeon can take the time needed to adjust the position of the emitter 9 exactly on the work position without particular problems (compatibly with the patient's anaesthesia time).

Opportunely, the source 8 has its power adjustable in order to adjust at will (at least within certain limits) the intensity of the signal 14 detectable from the exterior.

In general (except for a variation described below, which constitutes an exception), the signal 14 is essentially due to that part of electromagnetic radiation, exiting the emitter 9, that is transmitted through the bone tissues. When the non ionising electromagnetic radiation, exiting the emitter 9, has a spectrum distributed on a predetermined interval of wavelength selected in the range that goes from the near ultraviolet up to and including the mid infrared, the signal 14 is essentially determined by that fraction of electromagnetic radiation that, after exiting the emitter 9, traversed the cortex 12 of the bone 4 after undergoing absorption and/or diffusion phenomena by the bone tissues, the liquids and the soft parts encountered in its path from the emitter 9 towards the exterior. When the electromagnetic radiation exiting the emitter 9 is a part of the thermal infrared range, the transmitted radiation that determines the signal 14 has a partly different origin. The same liquids, soft parts and bone tissues, heated by the thermal radiation received from the emitter 9, in turn become sources of thermal radiation that is partly emitted towards the exterior, so that the intensity distribution of the signal 14 detectable from the exterior depends not only on absorption and diffusion phenomena, but also on phenomena of heat transfer in biological tissues.

In both cases, anyway, the operating principle is based on an appropriate illumination of the predetermined hole 5a from the interior of the nail 1 with a beam of electromagnetic radiation (at long, i.e. non ionising, waves), and on the detection of the radiation transmitted through the bone tissue in an area surrounding the incidence point. The point in which the bone 4 is to be drilled for the insertion of the locking screw coincides with the centroid of the distribution of the transmitted radiation.

Advantageously, the emitter 9 can be structured in such a way that the centroid of the intensity distribution of the signal 14 detectable from the exterior coincides with an intensity peak 140, so that the point where the bone 4 is to be drilled for the insertion of the locking screw coincides with the maximum of the distribution of the transmitted radiation.

In particular, opportunely, in a preferred embodiment of the invention illustrated in FIG. 6, the emitter 9 comprises an optical collimation system 16 that collimates the electromagnetic radiation in a beam within a predetermined solid angle 17 centred on the optical system 16, the solid angle 17 being coaxial with the predetermined hole 5a when the emitter 9 is in the work position.

When, as generally occurs (and is preferable in many cases), the source 8 generates the non ionising electromagnetic radiation and the emitter 9 serves merely as a conduit, the transmission line 15 comprises a waveguide for transporting the electromagnetic radiation to the emitter 9.

If the emitter 9 also comprises the optical collimation system 16, then the latter is directly coupled to the waveguide. In this case, the optical system 16, coupled to the waveguide, conveniently comprises a collimating lens 18 and a deflector 19 that deflects the radiation coming from the waveguide in the solid angle 17. The deflector 19 can be a mirror (for instance inclined by 45° relative to the axis of the waveguide), or a prism with total internal reflection. The presence of the deflector 19 is generally opportune to diminish the risk that, as a result of a marked bending of the waveguide when the emitter 9 is oriented correctly in view of its insertion into the internal channel 2, there is either a heavy loss of intensity of the radiation exiting the emitter 9 or a breakage of the waveguide itself.

In general, the waveguide can comprise an optical fibre. This optical fibre can be either single-mode or multi-mode. The waveguide can also comprise a fibre bundle, depending on requirements. Given the low powers generally involved (with the exception constituted by the variation described farther on, wherein high powers are involved), optical fibres made of plastic material can also be used.

It is highly opportune to collimate the radiation exiting the emitter 9 when it is distributed on wavelengths for which propagation occurs only by direct irradiation, such as wavelengths belonging to the ultraviolet, visible or near infrared ranges. The bone tissue is diffusing. Thus generally, even when the emitter 9 comprises the optical collimation system 16, the radiation transmitted beyond the cortex 12 of the bone 4 ends up having a substantially Lambertian angular distribution. Starting from an emitter 9 from which egresses a radiation beam corresponding to a non negligible solid angle 17 (for instance that of a non collimated radiation), there is a marked (and, at the limit, an unacceptable) decrease in contrast due to attenuation by diffusion.

The apparatus can be provided with a detector 24 of the signal 14 and with a related image converter, which can be positioned externally to the bone 4, to identify the centroid of the intensity distribution of the signal 14 and to display its position. This viewing system can comprise an image converter, a television camera (visible or infrared), a photodiode or light detector system. It can comprise light multipliers and/or image intensifiers, or any optoelectronic system able to make visible and/or intensify or attenuate or appropriately filter the electromagnetic radiation transmitted through the bone tissue.

Advantageously, however, if the radiation exiting the emitter 9 is distributed over wavelengths belonging to the range of the visible spectrum (i.e. essentially between 380 nanometres and 750 nanometres), the surgeon can identify the centroid of the intensity distribution of the signal 14 by direct vision (possibility indicated by the eye symbol shown in FIG. 2), without any other kind of aid. This advantage can lead to favour the choice of a system that uses visible radiation rather than one that uses infrared radiation which, though less prone to diffusion by the traversed tissues, makes it necessary to use auxiliary viewing systems.

If the source 8 directly generates electromagnetic radiation, subsequently carried to the emitter 9 by a waveguide, the source 8 can be a common source of visible light of appropriate spectrum (or according to its own characteristic spectrum), for instance incandescence, or gas discharge, or halogen. In this case, the sources are generally broad band sources.

The emission spectrum of the source 8 can also be modified by placing in front of it appropriate chromatic filters that select a predetermined portion of the visible wavelength range.

Alternatively, it is possible to use narrow band sources, such as lasers or LEDs, either with continuous wave or pulsed (in this case, to increase the peak emission power), sources that can be produced with variable emission wavelength, according to the type of laser or LED, from near ultraviolet to near infrared (as well as mid infrared in the case of some lasers). It is easy to find, for instance, low power lasers, which are coupled well with single-mode optical fibres, which emit powers in the order of 5 mW in the visible range or 10 mW in the near infrared range (for instance on wavelengths in the order of about 1 micrometre).

Very widespread (and particularly useful, as we shall see) are low power He—Ne lasers, with emission wavelength in the red range, around 633 nanometres, which need only to be coupled to the waveguide or optical fibre. There are also (low power) semiconductor lasers (with wavelengths also in the red range) already coupled to one or more optical fibres. They are very simple and economical systems. There are semiconductor lasers with emission peak practically on any useful wavelength.

It is particularly advantageous for the electromagnetic radiation exiting the emitter 9 to have its spectrum distributed over visible wavelengths ranging between 600 nanometres and 700 nanometres.

The absorption characteristics of the tissues and of the liquids which the electromagnetic radiation itself must traverse in its path from the emitter 9 to the exterior of the bone 4 have a great influence on the generation of the signal 14 on the outer superficial portion 13 of the cortex 12 of the bone 4 and on its intensity.

The interactions of blood, of the liquids inside the endomedullary channel 3, of the soft tissues (and, in general, of every component in the endomedullary channel 3 and/or in the bone 4) with the various radiations, according to their wavelength, are very important.

It is necessary to consider the absorption spectrum of blood and/or of biological tissues in general (which is not flat, but has areas of greater absorption and a transmission/transparency window for wavelengths centred around red) combined with the spectrum of the bone and of the tissues.

In general, the main causes of absorption in biological tissues are due to: oxy-haemoglobin (present in arterial blood); deoxy-haemoglobin (present in venous blood); water and various liquids; lipids. Within bone tissues, things change a little due to the contribution and the presence of collagen and of other bone structures. The sum of all these effects produces a global absorption spectrum that exhibits, on average, a first minimum between 600 and 700 nanometres, which gives rise to a dark red colour, and a second minimum around 800 nanometres. For wavelengths of less than 600 nanometres (towards green), absorption tends to grow, as it does for wavelengths exceeding 800 nanometres. Around 900 nanometres, there is a new absorption peak mainly due to the presence of water.

This is why it is very advantageous to use a source 8 that emits visible light on wavelengths of between 600 and 700 nanometres (or a broad band source 8 appropriately provided, as it egresses towards the waveguide, with spectrally selective filters on this wavelength range).

Ideal sources for the present purposes are: He—Ne lasers (emission on wavelengths near 633 nanometres) or semiconductor lasers calibrated on red. In addition to being optimal from the spectral viewpoint, these sources exhibit a low scattering level and, hence, allow to attain an excellent compromise on the level of attenuation of the radiation by diffusion and absorption on the part of the traversed tissues.

To facilitate the identification of the centroid of the intensity distribution of the signal 14 detectable from the exterior, in particular when it coincides with the intensity peak 140, the apparatus of the invention further comprises a contrast enhancing device 21, which can be positioned on the axis 6a of the predetermined hole 5a externally to the bone 4 and acts on the radiation portion transmitted through the bone tissues, enhancing the contrast between the intensity peak 140 and a peripheral part 141 of the signal 13 and improving resolution in the identification of the position of the axis 6a on the predetermined hole 5a.

Opportunely the contrast enhancing device 21 comprises an attennuating filter 22 for attenuating the radiation intensity. Said attenuating filter 22 lets through only the central, generally more intense, part of the distribution of the signal intensity 14, enhancing the contrast factor and the resolution.

The attenuation factor of the attenuating filter 22 can be defined as the ratio of the intensity of the light incident on the attenuating filter 22 and the intensity of the light transmitted thereby. It can be defined as a spectral parameter, i.e. calculated at each individual wavelength, or as an integral parameter, i.e. defined relative to total intensity (over all wavelengths together) of the radiation. Opportunely, said attenuation factor can be made adjustable discretely (for instance by superposing in removable fashion multiple attenuating elements 220 to constitute a single attenuating filter 22, as shown schematically in FIG. 2), or continuously (for instance, using special filters obtained with electrochromic materials), to obtain an optimal contrast factor.

The contrast enhancing device 21 can be integrated in the detector 24 of the signal 14. In the preferred variation described herein, i.e. the one that uses visible radiation, since the signal is directly observable by the operator, it is advantageously possible to dispense with the detector 24 and the systems connected thereto. In this case the contrast enhancing device 21 can be integrated in a simple pair of eyeglasses, wearable by the surgeon, provided with seats for the insertion of one or more attenuating filters 22.

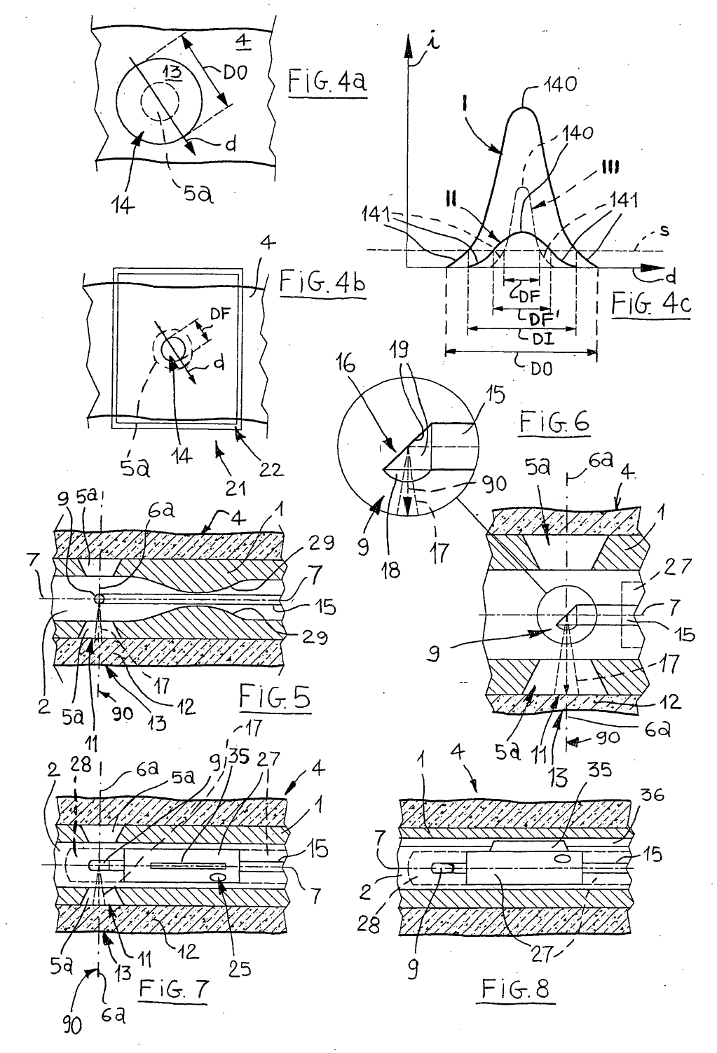

A principle according to which the use of such an attenuating filter 22 enhances the contrast between the intensity peak 140 and the peripheral part 141 of the signal 14 is schematically illustrated in FIGS. 4a, 4b, 4c (in which the represented components are illustrated only for purposes of explaining the principle, without regard to the actual dimensions and proportions).

The original distribution of the electromagnetic radiation which, egressing from the emitter 9, passes through the predetermined hole 5a, is modified traversing the cortex 12 of the bone 4 as a result of the multiple diffusion processes undergone through the bone tissue (and as a result of the absorption due mainly to liquids and soft tissues). When the radiation itself reaches the outer superficial portion 13 of the cortex 12, its distribution, as shown in FIG. 4a, is broadened over an area that as a first approximation has generally circular symmetry and has a diameter D0. In FIG. 4a said diameter D0 is shown greater than the diameter of the predetermined hole 5a (which, depending on the emission characteristics of the emitter 9, can frequently be the case). Moving on the outer superficial portion 13 along a straight line d superposed to a diameter of the approximately circular area whereon the signal 14 is distributed, the intensity i of the signal 14 varies. Its profile is generally the one schematically illustrated by the curve indicated as I in FIG. 4c.

Since the eye is easily saturated above a certain intensity threshold (indicated with the straight line s in FIG. 4c), the broadened circular area has an ample central area with diameter DI which is perceived as uniformly illuminated and whose centre is not identified. Attenuating the signal 14, depending on the characteristics of the attenuating filter 22, the intensity distribution, originally represented by the curve indicated as I, can be transformed for example into a distribution like those represented respectively by the curve indicated as II and by the curve indicated as III in FIG. 4c. The central area, perceived as uniform, is greatly reduced in diameter (as shown in FIG. 4c by the indication of the diameters DF and DF′, as well as in FIG. 4b, which illustrates the application of an attenuating filter 22 and its effect, compared to the initial configuration illustrated in FIG. 4a). Using appropriate attenuating filters 22 it is possible to reduce the diameter of the central area to the point of making visible only a small area corresponding to the axis 6a of the predetermined hole 5a wherefrom the light comes.

The attenuating filter 22 can comprise an attenuating element 220 of the “neutral density” type (i.e. with transmission spectrum substantially constant as a function of the wavelength) in the visible range. In combination with He—Ne lasers, attenuation factors between 1000 and 10000 (achievable with a single filter or with a combination of filters) yield good results.

To try to exploit the mechanisms of attenuation by absorption of the materials traversed by the radiation (and which have already been discussed in relation to the spectral characteristics of the radiation egressing from the emitter 9), it is also possible to use a spectrally profiled filter which allows the passage of the red radiation which (as stated also in relation to the sources) undergoes less diffusion/attenuation, allowing further to enhance contrast. In particular, the attenuating filter 22 can comprise an attenuating element 220 that attenuates the transmitted radiation whose wavelength is less than 600 nm. Preferably, the transmission spectrum of the attenuating element 220 has a maximum for wavelengths between 600 nm and 700 nm. In regard to this issue, it is necessary to note the importance of optimising both the spectral selectivity of the filter and (as the case may warrant) that of the emission spectrum of the source according to the absorption characteristics of the tissues and of the liquids which the radiation is to traverse.

Using, even with different sources, filters with spectrally selective transmission in these regions of the visible spectrum allows an additional, marked enhancement of contrast, eliminating all those components that are attenuated by the tissues and that only saturate the sensitivity of the eye.

Obviously, combinations of different types of attenuating elements 220 can be used (both “neutral density” and spectrally profiled, also with mutually different transmission spectra).

In a particular variation of the invention, opportunely, from the emitter 9 egresses electromagnetic radiation with a determined polarisation state and the attenuating filter 22 comprises a polariser element to attenuate to the highest possible extent the portion of radiation transmitted by the bone tissues not having the polarisation state of the radiation originally egressing from the emitter 9.

The diffusion of the electromagnetic radiation due mainly to bone tissues has a depolarising effect on the transmitted electromagnetic radiation. From the centre of the illuminated circular area, which is formed on the outer superficial portion 13 of the cortex 12 of the bone 4, emerges, towards the attenuating filter 22 the light that is most likely to reach the eye after traversing the bone without severe interference. Its polarisation state is generally very similar to the original one, whilst the light emerging from the periphery of the same area tends to be almost totally depolarised. The contrast enhancement is achieved, in fact, by highlighting the light that, having a very similar polarisation state to the original one, did not diffuse and, hence, is still collimated around the axis 6a of the predetermined hole 5a. The polarised light can be emitted directly by the source 8 or be obtained therefrom by placing an appropriate polariser filter between the source 8 and the waveguide.

Advantageously, provisions can be made to minimise the time required to adjust the position of the emitter 9 exactly on the working position, thereby reducing the probability of errors and the execution time (with the consequent reduction of the quantities of anaesthetic to be administered to the patient).

In particular, the apparatus according to the invention can comprise automatic positioning means for automatically positioning the emitter 9 at the height of the plane defined by the axis 6a of the predetermined hole 5a and by the axis 7 of the nail 1, acting at least when the emitter 9, inserted in the internal channel 2, is in proximity to the working position.

It is not strictly necessary for the emitter 9 also to be on the axis 7 of the nail 1, although it may be advantageous for it to be so in some cases. It is important that the emitter 9 be guided. Obviously, the emitter 9 itself could have dimensions corresponding to the diameter of the internal channel 2, in order to be positioned automatically, but normally the emitter 9 can be small or with a geometry not quite suitable to the purpose.

Advantageously, the automatic positioning means comprise means for centring the emitter 9 on the axis 7 of the nail 1, acting at least when the emitter 9, inserted in the internal channel 2, is in proximity to the working position.

In a first variation of the invention, illustrated in FIGS. 7, 8, 11a, 11b, said means for centring the emitter 9 on the axis 7 of the nail 1 comprise at least a widening 27 of the diameter of a sheath of the transmission line 15 at least in proximity to the emitter 9.

Opportunely, the widening 27 can be extended to enclose the emitter 9 with an element 28 that is transparent to the electromagnetic radiation egressing from the emitter 9 itself at least in the direction of the predetermined hole 5a. In this way, in addition to serving to protect the emitter 9, the transparent element 28 determines a minimisation of the thickness of potentially absorbing material that the electromagnetic radiation egressing from the emitter 9 must traverse.

In an alternative embodiment illustrated in FIG. 5, the means for centring the emitter 9 on the axis 7 of the nail 1 comprise, at least in proximity to the predetermined hole 5a, a narrowing 29 of the internal channel 2 around the axis 7 of the hole 1. Obviously, this solution can be adopted when sheath and emitter 9 have smaller diameter than that of the internal channel 2. Moreover, the narrowing 29 must have a minimum diameter at least corresponding to that of the guide wire, to allow the passage of the nail 1 on the guide wire and the subsequent extraction of the guide wire.

The presence of the narrowing 29 at least for a segment next to the holes 5a, 5b, 5c, 5d (at least next to the distal ones 5a, 5b, for which the present invention is particularly effective, if not next to all of them) prevents warping and allows a self-centering of the system on the axis 7 of the nail 1.

This solution is especially useful for tibial nails 1, that have a curved head (whereas femoral nails are generally straight).

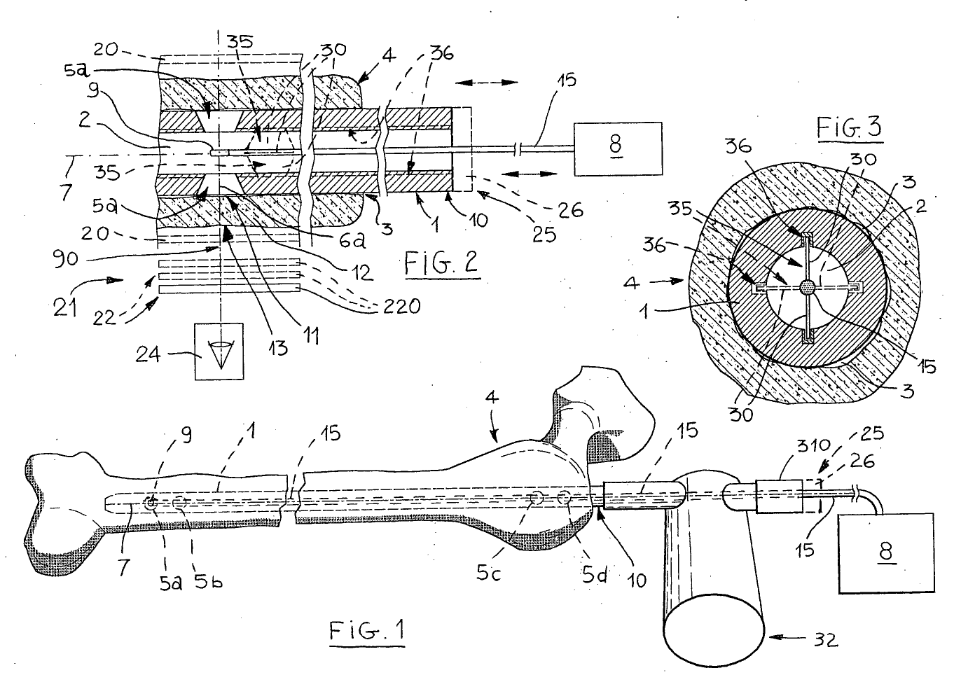

In an additional variation, shown in FIGS. 1 and 5, the means for centring the emitter 9 on the axis 7 of the nail 1 comprise at least two straps 30 oriented radially relative to the transmission line 15 and positioned along it in proximity to the emitter 9. Preferably, the straps 30 are at the same height along the transmission line 15. If there are more than two, they can be positioned on the various radii egressing from the transmission line 15.

To allow the exact, automatic identification of the working position along the axis 7 of the nail 1, the apparatus of the invention comprises an arrest reference 25 for stopping the insertion of the emitter 9 in the internal channel 2 when the emitter 9 reaches the working position. In combination with the centring means, the arrest reference 25 stops the insertion 2 of the emitter 9 and of a part of the transmission line 15 connected thereto into the internal channel exactly when the emitter 9 is positioned at the centre of the predetermined hole 5a.

At this point, in order for the emitter 9 to be correctly positioned in the working position it is sufficient to orient it in such a way that its emission axis 90 is parallel to the axis 6a of the predetermined hole 5a (if the emitter comprises the optical collimation system 16, said emission axis 90 coincides with the axis of the solid angle 17). For this purpose it may be sufficient to rotate the emitter 9 by acting on the sheath of the transmission line 15.

This can be made possible, for example, by providing at least the terminal segment of the transmission line 15 destined to be inserted into the internal channel 2 of a support element with high torsion constant. Said support element can be incorporated in the sheath or coincide therewith and be made of appropriate material, such as Kevlar. At least the terminal segment of the transmission line 15 destined to be inserted in the internal channel 2 can be enclosed by a substantially rigid sheath (for instance made of Kevlar or steel). At the end of the sheath can be positioned the emitter 9, possibly provided with the optical collimation system 16. It would be opportune for the diameter of the sheath to correspond with that of a normal guide wire.

In general, however, it may not be wholly easy to “seek” the correct orientation of the emitter 9 by twisting the sheath acting on its end accessible by the surgeon's hand (especially if an optical fibre and a flexible sheath are present), to attempt to make it rotate inside the internal channel 2. For the sake of practicality and of operating speed, it is absolutely preferable for the apparatus to comprise means for orienting the emitter 9 with certainty, acting in such a way that its axis of emission 90 is brought parallel to the axis 6a of the predetermined hole 5a. In particular, as shown in the figures, advantageously the apparatus of the invention can comprise means 34 of mutual interference between the terminal segment of the transmission line 15, destined to be inserted in the internal channel 2, and the wall of the internal channel 2 which means, by allowing the transmission line 15 to slide in the internal channel 5, determine the certain orientation of the emitter 9 with its axis of transmission 90 parallel to the axis 6a of the predetermined hole 5a at least in proximity thereto. In a possible embodiment, shown in the figures, the mutual interference means 34 comprise a fin 35 positioned on the transmission line 15 in proximity to the emitter 9 and able to be inserted in sliding fashion at least with one of its ends in a corresponding guiding groove 36 obtained on the surface of the internal channel 2.

More than one fin 35 can be provided. In this case the fins 35 can also coincide with the straps 30 for centring on the axis 7 of the nail 1.

Opportunely, in an embodiment of the invention, the arrest reference 25 comprises stopping elements 33 for stopping the motion of the transmission line 15 in the nail 1, when the emitter 9 reaches the working position, acting between the transmission line 15 and the wall of the internal channel and able to be elastically disengaged, in particular to allow a further motion of the emitter 9 towards a different working position corresponding to another one of the transverse through holes 5a, 5b, 5c, 5d or to extract the emitter 9 from the nail 1. The stopping elements 33 can comprise an expandable head positioned on the emitter 9, destined to be inserted into a seat opportunely obtained in the wall of the internal channel 2.

Alternatively or in combination, the arrest reference 25 comprises an abutment element 26, able to abut the proximal end 10 of the nail 1 or its extension 100 and able to be positioned along the transmission line 15 in a position of arrest of the insertion of the transmission line 15 in the internal channel 2 corresponding to the working position of the emitter 9.

In general, the position of the abutment element 26 can obviously be adjusted according to the hole 5a, 5b, 5c, 5d to be reached and to the length of the nail 1. It can be made coaxial, in sliding fashion, to the sheath of the transmission line 15.

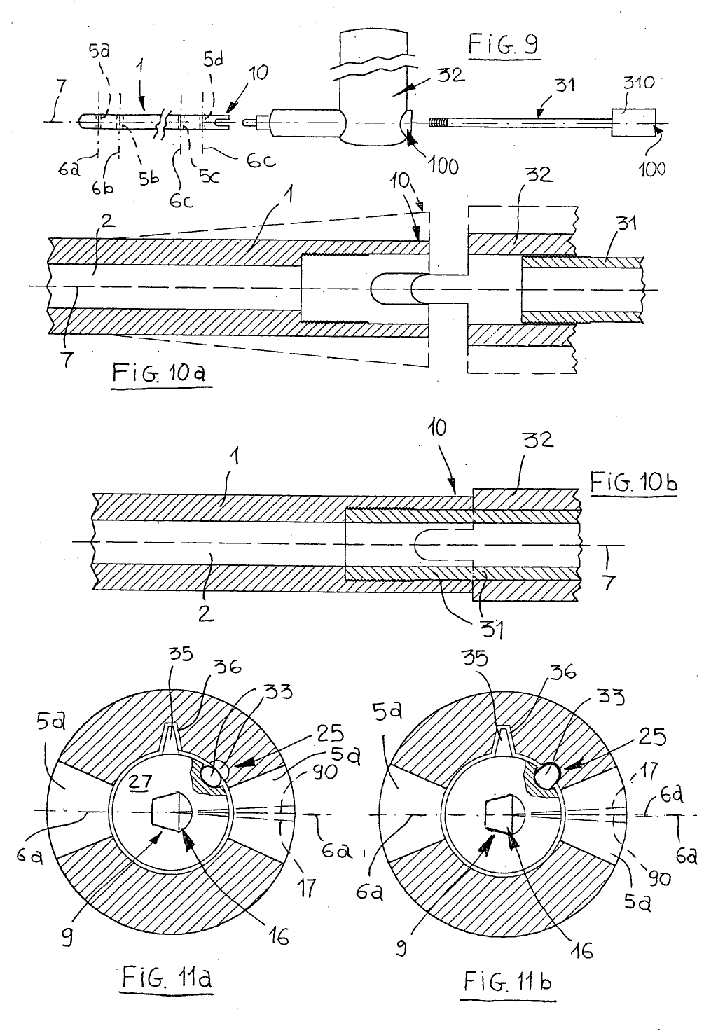

The extension 100 can be a part of a hand piece 32, opportunely associated to the nail 1 in correspondence with the proximal end 10, said part having a passage channel for the emitter 9 and for the corresponding transmission line 15 communicating with the internal channel 2. Alternatively, the extension 100 can also be the head 310 of a fastening pin 31, also tubular to allow the passage of the emitter 9 and of the corresponding transmission line 15, able to be coaxially associated to the nail 1 (and to the hand piece 32, if present) in a known manner. Obviously, as is well known, the hand piece 32 and the corresponding fastening pin 31 must have an inner diameter which allows the passage of at least a guide wire. In partucular, preferably, the apparatus of the invention comprises a fastening pin 31 for fastening a tubular hand piece 32 to the nail 1, the fastening pin 31 being tubular, coaxial to the nail 1 and with its inner diameter corresponding to the diameter of the internal channel 2 of the nail 1. In this latter case, especially in case of nails 1 with small diameter, it may be necessary, to allow the insertion of the fastening pin 31, to have a gradual widening of the nail 1 in proximity to its proximal end 10 (see the dashed line in FIG. 10a). These characteristics are illustrated in FIGS. 9, 10a and 10b.

Use of the abutment element 26 is particularly suitable when the sheath is rigid. In an embodiment of the invention, at least the terminal segment of the transmission line 15 destined to be inserted in the internal channel 2 is enclosed by a substantially rigid sheath and the abutment element 26 and the proximal end 10 of the nail 1 or its extension 100 comprise mutual engagement means for the certain orientation of the emitter 9 with its axis of emission 90 parallel to the axis 6a of the predetermined hole 5a.

Said mutual engagement means can be of various kinds, for instance pins and corresponding seats, or appropriate contoured abutments.

In an embodiment of the invention, the nail 1 comprises, already integrated, emitters 9, already opportunely positioned and aligned at least in front of corresponding distal holes 5a, 5b (optionally also in front of proximal holes 5c and 5d) and provided with corresponding segments of transmission line 15 also integrated in the nail 1 and provided, at their opposite end relative to the emitters 9, with connectors to corresponding extensions of the transmission line 15 to the source 8. The system can thus be readied before inserting the nail 1 on the guide wire in the endomedullary channel 3, integrating all necessary elements in its interior. The nail 1 could also be obtained with single-use integrated elements.

This solution would eliminate every problem of centring, aligning and orienting the emitter 9 relative to the selected hole, since all would already be pre-set.

In a variation of the invention, which can also comprise all (or only some) of the positioning, centring and aligning systems discussed heretofore, the source 8 comprises an electrical power generator, the emitter 9 comprises a heating element and the transmission line 15 comprises an electrical wire connecting the heating element with the electrical power generator, the heating element emitting, when excited by the electrical power, electromagnetic radiation at wavelengths in the thermal infrared region.

The source 8 can be able to provide current continuously or variably at a predetermined voltage, with adjustable power, to supply it, on the emitter 9, to an electrical impedance or a resistor. The transmission line 15 can comprise an appropriate electrical wire, or coaxial cable, or electromagnetic guide, able to transmit electrical power from the source 8 to the emitter 9. In particular, it is opportune for the thermal emitter 9 constituted by the heating element to be as point-like as possible relative to the dimensions of the hole into which the locking screw is to be driven. As discussed elsewhere, in this case the propagation of radiation in the tissues is coupled with other phenomena of thermal radiation emission from the tissues, due to their heating, and it is therefore also linked to phenomena of heat transport by thermal conduction, thermal convection, irradiation—direct and otherwise—in the liquid inside the bone, in the tissues and in the bone.

A particular variation of the invention generates a signal 14 outside the bone 4 which is reduced to a physical characteristic produced on the bone 4 itself. In particular, alternatively to what has been discussed heretofore, the source 8 comprises a surgical power laser and the emitter 9 comprises an optical focusing system coupled to the transmission line 15, which, when the emitter 9 is in the working position, directs and focuses the laser beam on a part of bone tissue in correspondence with the axis 6a of the predetermined hole 5a. The signal 14 is hence defined by the perforation of the bone effected with the laser beam. The apparatus further comprises protection screens 20, able to be positioned in removable fashion around the fractured part to protect the operator from the action of the laser beam. The direct use of a surgical laser to generate a physical (not optical) signal, i.e. a perforation, leads to safety problems: it is a Class 4 power laser. In particular, the surgical laser acts in this case from the interior of the nail 1 outwardly, being dangerous also in the operating room, even if defocused by the diffusive effect of the bone tissue. Once the bone is holed, the laser beam propagates free in the operating room. This is why the protection screens 20 around the operated part (leg, arm, etc.) are necessary. Therefore, it is also obviously necessary to insert and position the emitter 9 blindly, unless one provides an auxiliary light source that illuminates the predetermined hole 5a from the interior, in ways already discussed for the previous embodiments, and that acts with the surgical laser off (which is complicated, however). Thus, in this variation it is generally also necessary to provide the mechanical systems for exactly guiding, positioning and orienting the emitter 9 in correspondence with the predetermined hole 5a discussed above for similar systems.

A surgical laser can be CO₂ (emission wavelength of about 10 micrometres) or neodymium (emission wavelength of about 1 micrometre). It complicates the system for the reasons described above and because it makes it necessary not only to collimate, but also to focus the beam on the bone. The system can be costly.

On the contrary, a common low power He/Ne laser needs only be coupled to its optical fibre. There are, as stated, (low power) semiconductor lasers (with wavelengths also in the red region) already connected in fibres. These systems are far simpler and economical.

The invention achieves important advantages. It considerably improves on the current technique of driving the locking screws, especially the distal ones. It allows totally to eliminate irradiation with dangerous ionising radiation during the nail locking phase. It makes the operation easy and precise. It allows to use components that, in the preferred embodiments, are simple to manufacture or easy to obtain. The purely mechanical part of the apparatus allows to make the implantation of the endomedullary nail easier, quicker and more precise. Surgical and, consequently, anaesthetic times are drastically reduced. A high precision level in the driving of the locking screws (especially the distal ones) can be achieved in a simple fashion. The simplification of the surgical instrumentation determines a consequent reduction in material production, maintenance, sterilisation and storage costs.

The invention thus conceived can be subject to numerous modifications and variations, without thereby departing from the scope of the inventive concept that characterises it.

Moreover, all components can be replaced by other technically equivalent elements.

In practice all materials used, as well as the shapes and dimensions of the various components, can be any depending on requirements.

1. Apparatus for the osteosynthesis of bone fractures by means of locked endomedullary nailing, of the type comprising:
a tubular nail defining an internal coaxial through channel, provided with transverse through holes whose axis intersects the axis of the nail and able to be inserted in a medullar channel of a bone;
a device for locating the axis of a predetermined hole selected among said holes, along which the bone is to be drilled to drive a corresponding screw for locking the nail on the bone;
wherein the locating device comprises:
a source of electromagnetic power;
an emitter of the electromagnetic power in the form of non ionising electromagnetic radiation, able to be inserted inside the nail along the internal channel from a proximal end of the nail at least to a working position, located on the axis of the predetermined hole, in which at least part of the non ionising electromagnetic radiation is directed from the emitter, through the predetermined hole, on an inner superficial portion of the cortex of the bone corresponding to the axis of the predetermined hole and generates, beyond an outer superficial portion of the cortex of the bone, also corresponding to the axis of the predetermined hole, a signal detectable from the exterior having an intensity distribution with its centroid in correspondence with the axis of the predetermined hole; a line for transmitting the electromagnetic power from the source to the emitter.

2. Apparatus as claimed in claim 1, wherein the centroid of the intensity distribution of the signal detectable from the exterior coincides with an intensity peak.

3. Apparatus as claimed in claim 1, wherein the non ionising electromagnetic radiation egressing from the emitter has its spectrum distributed on a predetermined interval of wavelengths selected in the range that goes from near ultraviolet to mid infrared, ends included, the signal being essentially determined by the radiation portion transmitted through the bone tissues.

4. Apparatus as claimed in claim 3, wherein the electromagnetic radiation egressing from the emitter is visible light.

5. Apparatus as claimed in claim 4, wherein the wavelengths belonging to the predetermined interval are comprised between 600 nanometres and 700 nanometres.

6. Apparatus as claimed in claim 3, wherein the wavelengths belonging to the predetermined interval are comprised in the infrared range.

7. Apparatus as claimed in claim 3, wherein the emitter comprises an optical collimation system that collimates the electromagnetic radiation in a beam within a predetermined solid angle centred on the optical system, the solid angle being coaxial with the predetermined hole when the emitter is in the working position.

8. Apparatus as claimed in claim 4, wherein the emitter comprises an optical collimation system that collimates the electromagnetic radiation in a beam within a predetermined solid angle centred on the optical system, the solid angle being coaxial with the predetermined hole when the emitter is in the working position.

9. Apparatus as claimed in claim 3, wherein the source directly generates the non ionising electromagnetic radiation, the transmission line comprising a waveguide for carrying the electromagnetic radiation to the emitter.

10. Apparatus as claimed in claim 7, wherein the source directly generates the non ionising electromagnetic radiation, wherein the transmission line comprises a waveguide for carrying the electromagnetic radiation to the emitter and wherein the optical system, coupled to the waveguide, comprises a collimating lens and a deflector that deflects the radiation coming from the waveguide in the solid angle.

11. Apparatus as claimed in claim 1, wherein the source comprises an electrical power generator, the emitter comprises a heating element and the transmission line comprises an electrical wire connecting the heating element to the electrical power generator, the heating element emitting, when excited by the electrical power, electromagnetic radiation at wavelengths in the thermal infrared region.

12. Apparatus as claimed in claim 1, wherein the source comprises a surgical power laser and the emitter comprises a focusing optical system coupled with the transmission line, which, when the emitter is in the working position, directs and focuses the laser beam on a part of bone tissue in correspondence with the axis of the predetermined hole, the signal being defined by the perforation of the bone tissue operated with the laser beam, the apparatus further comprising protecting screens, able to be positioned in removable fashion around the fractured part to protect the operator from the action of the laser beam.

13. Apparatus as claimed in claim 3, wherein a centroid of the intensity distribution of the signal detectable from the exterior coincides with an intensity peak and in that the apparatus further comprises a contrast enhancement device, which can be positioned on the axis of the predetermined hole externally to the bone and acts on the portion of radiation transmitted through the bone tissues enhancing the contrast between the intensity peak and a peripheral part of the signal and improving the resolution in the identification of the position of the axis of the predetermined hole.

14. Apparatus as claimed in claim 13, wherein the contrast enhancement device comprises an attenuating filter for attenuating the intensity of the radiation.

15. Apparatus as claimed in claim 14, characterised in that the attenuating filter comprises an attenuating element of the type with “neutral density” in the visible range.

16. Apparatus as claimed in claim 14, wherein the attenuating filter comprises an attenuating element that attenuates the transmission radiation whose wavelength is less than 600 nm.

17. Apparatus as claimed in claim 16, wherein the transmission spectrum of the attenuating element has a maximum for wavelengths between 600 nm and 700 nm.

18. Apparatus as claimed in claim 14, wherein the attenuating filter has an adjustable attenuation factor.

19. Apparatus as claimed in claim 14, wherein from the emitter egresses electromagnetic radiation with a predetermined polarisation state and wherein the attenuating filter comprises a polariser element to attenuate the portion of radiation transmitted by the bone tissues not having the polarisation state of the radiation originally egressing from the emitter.

20. Apparatus as claimed in claim 1, which further comprises a detector of the signal and a related image converter, able to be positioned externally to the bone for identifying the centroid of the intensity distribution of the signal and visualising its position.

21. Apparatus as claimed in claim 1, which further comprises automatic positioning means for automatically positioning the emitter at the height of the plane defined by the axis of the predetermined hole and by the axis of the nail, acting at least when the emitter, inserted in the internal channel, is in proximity to the working position.

22. Apparatus as claimed in claim 21, wherein the automatic positioning means comprise means for centring the emitter on the axis of the nail acting at least when the emitter, inserted in the internal channel, is in proximity to the working position.

23. Apparatus as claimed in claim 22, wherein the means for centring the emitter of the axis of the nail comprise at least a widening of the diameter of a sheath of the transmission line at least in proximity to the emitter.

24. Apparatus as claimed in claim 23 wherein the widening extends to enclose the emitter with an element transparent to the electromagnetic radiation egressing from the emitter itself at least in the direction of the predetermined hole.

25. Apparatus as claimed in claim 22, wherein the means for centring the emitter on the axis of the nail comprise, at least in proximity to the predetermined hole, a narrowing of the internal channel around the axis of the nail.

26. Apparatus as claimed in claim 22, wherein the means for centring the emitter on the axis of the nail comprise at least two straps oriented radially relative to the transmission line and positioned along it in proximity to the emitter.

27. Apparatus as claimed in claim 21, which further comprises an arrest reference for arresting the insertion of the emitter in the internal channel when the emitter reaches a working position.

28. Apparatus as claimed in claim 22, which further comprises an arrest reference for arresting the insertion of the emitter in the internal channel when the emitter reaches a working position.

29. Apparatus as claimed in claim 27, wherein the arrest reference comprises elements for stopping the motion of the transmission line in the nail when the emitter reaches the working position, acting between the transmission line and the wall of the internal channel and able to be elastically disengaged.

30. Apparatus as claimed in claim 28, wherein the arrest reference comprises elements for stopping the motion of the transmission line in the nail when the emitter reaches the working position, acting between the transmission line and the wall of the internal channel and able to be elastically disengaged.

31. Apparatus as claimed in claim 27, wherein the arrest reference comprises an abutment element, able to abut the proximal end of the nail or an extension thereof and able to be positioned along the transmission line in a position of arrest of the insertion of the transmission line in the internal channel corresponding to the working position of the emitter.

32. Apparatus as claimed in claim 28, wherein the arrest reference comprises an abutment element, able to abut the proximal end of the nail or an extension thereof and able to be positioned along the transmission line in a position of arrest of the insertion of the transmission line in the internal channel corresponding to the working position of the emitter.

33. Apparatus as claimed in claim 31, wherein the terminal segment of the transmission line destined to be inserted in the internal channel is enclosed by a substantially rigid sheath and wherein the abutment element and the proximal end of the nail or its extension comprise mutual engagement means for the certain orientation of the emitter with an axis of emission thereof parallel to the axis of the predetermined hole.

34. Apparatus as claimed in claim 32, wherein the terminal segment of the transmission line destined to be inserted in the internal channel is enclosed by a substantially rigid sheath and wherein the abutment element and the proximal end of the nail or its extension comprise mutual engagement means for the certain orientation of the emitter with an axis of emission thereof parallel to the axis of the predetermined hole.

35. Apparatus as claimed in claim 21, which further comprises means for the certain orientation of the emitter with an axis of emission thereof parallel to the axis of the predetermined hole at least in proximity thereto.

36. Apparatus as claimed in claim 22, which further comprises means for the certain orientation of the emitter with an axis of emission thereof parallel to the axis of the predetermined hole at least in proximity thereto.

37. Apparatus as claimed in claim 27, which further comprises means for the certain orientation of the emitter with an axis of emission thereof parallel to the axis of the predetermined hole at least in proximity thereto.

38. Apparatus as claimed in claim 28, which further comprises means for the certain orientation of the emitter with an axis of emission thereof parallel to the axis of the predetermined hole at least in proximity thereto.

39. Apparatus as claimed in claim 35, wherein the means for the certain orientation of the emitter comprise means of mutual interference between the terminal segment of the transmission line, destined to the be inserted in the internal channel, and the wall of the internal channel, which determine the orientation of the emitter allowing the transmission line to slide in the internal channel.

40. Apparatus as claimed in claim 36, wherein the means for the certain orientation of the emitter comprise means of mutual interference between the terminal segment of the transmission line, destined to the be inserted in the internal channel, and the wall of the internal channel, which determine the orientation of the emitter allowing the transmission line to slide in the internal channel.

41. Apparatus as claimed in claim 37, wherein the means for the certain orientation of the emitter comprise means of mutual interference between the terminal segment of the transmission line, destined to the be inserted in the internal channel, and the wall of the internal channel, which determine the orientation of the emitter allowing the transmission line to slide in the internal channel.

42. Apparatus as claimed in claim 38, wherein the means for the certain orientation of the emitter comprise means of mutual interference between the terminal segment of the transmission line, destined to the be inserted in the internal channel, and the wall of the internal channel, which determine the orientation of the emitter allowing the transmission line to slide in the internal channel.

43. Apparatus as claimed in claim 39, wherein the means of mutual interference means comprise a fin positioned on the transmission line in proximity to the emitter and able to be inserted in sliding fashion at least with one of its ends in a corresponding guiding groove obtained on the surface of the internal channel.

44. Apparatus as claimed in claim 40, wherein the means of mutual interference means comprise a fin positioned on the transmission line in proximity to the emitter and able to be inserted in sliding fashion at least with one of its ends in a corresponding guiding groove obtained on the surface of the internal channel.

45. Apparatus as claimed in claim 41, wherein the means of mutual interference means comprise a fin positioned on the transmission line in proximity to the emitter and able to be inserted in sliding fashion at least with one of its ends in a corresponding guiding groove obtained on the surface of the internal channel.

46. Apparatus as claimed in claim 42, wherein the means of mutual interference means comprise a fin positioned on the transmission line in proximity to the emitter and able to be inserted in sliding fashion at least with one of its ends in a corresponding guiding groove obtained on the surface of the internal channel.

47. Apparatus as claimed in claim 21, wherein at least the terminal segment of the transmission line destined to be inserted in the internal channel comprises a support element with high torsion constant.

48. Apparatus as claimed in claim 22, wherein at least the terminal segment of the transmission line destined to be inserted in the internal channel comprises a support element with high torsion constant.

49. Apparatus as claimed in claim 27, wherein at least the terminal segment of the transmission line destined to be inserted in the internal channel comprises a support element with high torsion constant.

50. Apparatus as claimed in claim 28, wherein at least the terminal segment of the transmission line destined to be inserted in the internal channel comprises a support element with high torsion constant.

51. Apparatus as claimed in claim 35, wherein at least the terminal segment of the transmission line destined to be inserted in the internal channel comprises a support element with high torsion constant.

52. Apparatus as claimed in claim 36, wherein at least the terminal segment of the transmission line destined to be inserted in the internal channel comprises a support element with high torsion constant.

53. Apparatus as claimed in claim 37, wherein at least the terminal segment of the transmission line destined to be inserted in the internal channel comprises a support element with high torsion constant.

54. Apparatus as claimed in claim 38, wherein at least the terminal segment of the transmission line destined to be inserted in the internal channel comprises a support element with high torsion constant.

55. Apparatus as claimed in claim 21, wherein at least the terminal segment of the transmission line destined to be inserted in the internal channel is enclosed by a substantially rigid sheath.

56. Apparatus as claimed in claim 22, wherein at least the terminal segment of the transmission line destined to be inserted in the internal channel is enclosed by a substantially rigid sheath.

57. Apparatus as claimed in claim 27, wherein at least the terminal segment of the transmission line destined to be inserted in the internal channel is enclosed by a substantially rigid sheath.

58. Apparatus as claimed in claim 28, wherein at least the terminal segment of the transmission line destined to be inserted in the internal channel is enclosed by a substantially rigid sheath.

59. Apparatus as claimed in claim 35, wherein at least the terminal segment of the transmission line destined to be inserted in the internal channel is enclosed by a substantially rigid sheath.

60. Apparatus as claimed in claim 36, wherein at least the terminal segment of the transmission line destined to be inserted in the internal channel is enclosed by a substantially rigid sheath.

61. Apparatus as claimed in claim 37, wherein at least the terminal segment of the transmission line destined to be inserted in the internal channel is enclosed by a substantially rigid sheath.

62. Apparatus as claimed in claim 38, wherein at least the terminal segment of the transmission line destined to be inserted in the internal channel is enclosed by a substantially rigid sheath.

63. Apparatus as claimed in claim 1, which further comprises a fastening pin for fastening a tubular hand piece to the nail, the fastening pin being tubular, coaxial to the nail and with its inner diameter corresponding to the diameter of the internal channel of the nail.

64. Apparatus as claimed in claim 1, wherein the nail comprises emitters, already appropriately positioned and aligned at least in front of corresponding distal holes, and corresponding segments of transmission line, also integrated in the nail and provided, at an opposite end relative to the emitters, with connectors to corresponding extensions of the transmission line through to the source.

65. Apparatus as claimed in claim 5, wherein the emitter comprises an optical collimation system that collimates the electromagnetic radiation in a beam within a predetermined solid angle centred on the optical system, the solid angle being coaxial with the predetermined hole when the emitter is in the working position.

66. Apparatus as claimed in claim 7, wherein the source directly generates the non ionising electromagnetic radiation, the transmission line comprising a waveguide for carrying the electromagnetic radiation to the emitter.
